(19) United States
(12) Patent Application Publication
(10) Pub. No.: US 2014/0308251 A1
(43) Pub. Date: Oct. 16, 2014

(54) INTERVERTEBRAL DISC REPAIR, METHODS AND DEVICES THEREFOR

(71) Applicant: ISTO TECHNOLOGIES, INC., ST. LOUIS, MO (US)

(72) Inventors: Mitchell S. Seyedin, Monte Sereno, CA (US); H. Davis Adkisson, St. Louis, MO (US)

(73) Assignee: ISTO TECHNOLOGIES, INC., ST. LOUIS, MO (US)

(21) Appl. No.: 14/218,486

(22) Filed: Mar. 18, 2014

Related U.S. Application Data
(63) Continuation of application No. 11/458,278, filed on Jul. 18, 2006.
Application No. 11/458,278 is a continuation-in-part of application No. 11/063,183, filed on Feb. 22, 2005, now Pat. No. 7,879,604.
(60) Provisional application No. 60/546,619, filed on Feb. 20, 2004.

(51) Int. Cl. A61K 35/32; A61K 38/36; A61K 38/48

(52) U.S. Cl. CPC A61K 35/32; A61K 38/4833; A61K 38/36
USPC 424/93.7

(57) ABSTRACT

The present application discloses compositions, methods and devices for treatment of a degenerative intervertebral disc. A composition can comprise chondrocytes expressing type II collagen. These chondrocytes can be obtained from human cadavers up to about two weeks following death, and can be grown in vitro. The compositions can further comprise one or more biocompatible molecules. Treatment of a degenerative disc can comprise injecting or implanting a composition comprising the chondrocytes into a degenerative disc through an aperture or incision. If the aperture or incision is closed with a suture or a glue after introduction of the chondrocytes, the closure can withstand over 400 N of compression force.

### CROSS-REFERENCE TO RELATED APPLICATIONS

This application is a Continuation of application Ser. No. 11/458,278, filed Jul. 18, 2006, which is a Continuation-in part of application Ser. No. 11/063,183, filed Feb. 22, 2005, which claims benefit of U.S. Provisional Application No. 60/546,619 filed Feb. 20, 2004, the disclosures of all of which are incorporated herein by reference in their entireties.

### INTRODUCTION

Intervertebral disc degeneration is a leading cause of pain and disability in the adult population. Approximately 80% of the population experience at least a single episode of significant back pain in their lifetimes. For many individuals, spinal disorders become a lifelong affliction. The morbidity associated with disc degeneration and its spectrum of associated spinal disorders is responsible for significant health care, economic and social costs. Furthermore, changes in disc morphology, such as disc compression associated with aging, can lead to unwanted changes in height or posture. Current treatments for repairing or ameliorating disc degeneration, such as spinal fusion, can be expensive, painful, or lengthy. Alternative treatments are, therefore, needed.

### SUMMARY

In view of the need for disc degeneration treatments, the present inventors have devised compositions, methods and devices for repair, replacement and/or supplementation of an intervertebral disc which involve implantation or injection of chondrocytes into a degenerative disc, as well as compositions and methods for providing chondrocytes to a treatment provider.

Some embodiments of the present teachings include methods of repairing a degenerative intervertebral disc in a human patient in need of treatment. In these embodiments, a method can comprise implanting, into the intervertebral disc, chondrocytes obtained from a cadaver. The cadaver chondrocytes can be from any cartilaginous tissue of the cadaver, provided the chondrocytes express type II collagen. Furthermore, the chondrocytes expressing type II collagen can be chondrocytes expressing high molecular weight sulfated proteoglycan (HSPG). The chondrocytes can be, for example, hyaline cartilage chondrocytes. In various configurations, the chondrocytes can be chondrocytes from one or more intervertebral discs, or the chondrocytes can be non-intervertebral disc chondrocytes. Chondrocytes from an intervertebral disc can be chondrocytes from the annulus of a disc, chondrocytes from the nucleus pulposus of a disc, or a combination thereof. Non-limiting examples of non-intervertebral disc tissue which can be sources of chondrocytes include cartilage of the nose, ears, trachea and larynx, as well as articular cartilage, costal cartilage, cartilage of an epiphyseal plate, and combinations thereof.

In various aspects of the present teachings, the chondrocytes can be extracted from a cadaver at any time following death while the chondrocytes remain viable. In various configurations, chondrocytes can be extracted from a cadaver up to about fourteen days following death. Chondrocytes can be removed from a cadaver from about one hour following death to about fourteen days following death, from greater than 24 hours following death to about thirteen days following death, from about two days following death to about twelve days following death, from about three days following death to about twelve days following death, or from about four days following death to about ten days following death.

In some embodiments, chondrocytes of the present teachings can be chondrocytes extracted from a cadaver of any chronological age at time of death. In various configurations, chondrocytes can be extracted from a cadaver which is no older than about 40 years of age at time of death, no older than about 30 years of age at time of death, no older than about 20 years of age at time of death, or no older than about 10 years of age at time of death. A donor cadaver need not be a familial member of a recipient, or be otherwise matched immunologically.

In various embodiments, chondrocytes which are extracted from a cadaver can be grown in vitro prior to their implantation or injection into a recipient patient or purveyance to a treatment provider. Growth of chondrocytes in vitro can be used, for example, to increase the number of chondrocytes available for implantation or injection. In non-limiting example, chondrocyte numbers can be increased about two fold or greater, about ten fold or greater, or about twenty fold or greater. In various configurations, growing chondrocytes in vitro can comprise placing one or more cartilage tissue pieces removed from a cadaver into a tissue culture or cell culture medium which comprises nutrients, buffers, salts, proteins, vitamins and/or growth factors which promote chondrocyte growth, and incubating the chondrocytes. In certain configurations, tissue comprising chondrocytes expressing type II collagen can be dissociated into single cells or small groups of cells prior to, or in conjunction with, their introduction into a culture medium. In addition, in some aspects, in vitro culture of chondrocytes expressing type II collagen can further comprise removing non-chondrocyte cells from a cell- or tissue-culture.

In various embodiments of the present teachings, chondrocytes expressing type II collagen can be comprised by a composition which can be implanted or injected into an intervertebral disc of a patient in need of treatment. Accordingly, in certain embodiments, the present teachings also include compositions comprising cadaver chondrocytes expressing type II collagen for use in implantation or injection into a degenerative intervertebral disc of a patient in need of treatment. In some configurations of these embodiments, the chondrocytes of these compositions can comprise chondrocytes expressing high molecular weight sulfated proteoglycan. In some configurations, a composition comprising chondrocytes expressing type II collagen can further comprise at least one biocompatible molecule. Non-limiting examples of biocompatible molecules which can be comprised by a composition of the present teachings include fibrinogen, fibrin, thrombin, type I collagen, type II collagen, type III collagen, fibronectin, laminin, hyaluronic acid (HA), hydrogel, pegylated hydrogel, chitosan, and combinations thereof.

In various embodiments, the present teachings include methods of forming a composition comprising cadaver chondrocytes. A composition formed by these methods can further comprise one or more biocompatible molecules such as those described supra. Accordingly, methods of these embodiments can comprise contacting cadaver chondrocytes expressing type II collagen with one or more biocompatible molecules, such as, for example, fibrinogen, fibrin, thrombin, type I collagen, type II collagen, type III collagen, fibronectin, laminin, hyaluronic acid, hydrogel, pegylated hydrogel, chitosan and combinations thereof. The cadaver chondrocytes expressing type II collagen can be, in some configurations, chondrocytes which also express high molecular weight sulfated proteoglycan. In certain aspects, the chondrocytes can be incubated in vitro in a culture medium prior to the contacting with one or more biocompatible molecules.

In various embodiments, the present teachings include methods of forming a composition comprising cadaver tissue comprising chondrocytes. A composition formed by these methods can further comprise one or more biocompatible molecules such as those described supra. Accordingly, methods of these embodiments can comprise contacting cadaver tissue comprising chondrocytes expressing type II collagen with one or more biocompatible molecules, such as, for example, fibrinogen, fibrin, thrombin, type I collagen, type II collagen, type III collagen, fibronectin, laminin, hyaluronic acid, hydrogel, pegylated hydrogel, chitosan and combinations thereof. The cadaver chondrocytes expressing type II collagen can be, in some configurations of these embodiments, chondrocytes which also express high molecular weight sulfated proteoglycan. In certain aspects of these embodiments, cadaver tissue comprising chondrocytes expressing type II collagen can be incubated in vitro in a culture medium prior to the contacting with one or more biocompatible molecules.

In various aspects of the present teachings, a composition comprising both cadaver chondrocytes expressing type II collagen and one or more biocompatible molecules can be implanted or injected into a degenerative intervertebral disc in a patient in need of treatment. In various aspects, implantation or injection of a composition into a disc can comprise implantation or injection of the composition into the annulus of the disc, implantation or injection of the composition into the nucleus pulposus of the disc, implantation or injection of the composition into one or both endplates of the disc, or a combination thereof. In some configurations, an aperture can be formed in an annulus of a degenerative disc, and a composition can be introduced into the disc through the aperture. In some configurations, surgical techniques such as vertebroplasty and kyphoplasty (Garfin, S. R., et al., Spine 26: 1511-1515, 2001) can be adapted or modified for introducing chondrocytes into a degenerative disc of a patient.

In various embodiments, the present teachings include an apparatus configured for injection of chondrocytes expressing type II collagen to an intervertebral disc of a patient in need of treatment. An apparatus configured for injection of chondrocytes expressing type II collagen into an intervertebral disc can comprise chondrocytes expressing type II collagen. Chondrocytes of these embodiments can comprise chondrocytes expressing high molecular weight sulfated proteoglycan. In various configurations, the apparatus can comprise a composition which comprises the chondrocytes and at least one biocompatible molecule, such as, for example, a biocompatible molecule described supra. In certain embodiments, the chondrocytes expressing type II collagen comprised by the apparatus can be cadaver chondrocytes. The cadaver chondrocytes in these embodiments can be intervertebral disc chondrocytes, or non-intervertebral disc chondrocytes, such as those described supra. In some configurations of these embodiments, the chondrocytes can be comprised by cadaver tissue. An apparatus of the present teachings can further comprise, in some configurations, a syringe, a double syringe, a hollow tube, such as a hollow needle (for example, a Jamshidi needle), a cannula, a catheter, a trocar, a stylet, an obturator, or other instruments, needles or probes for cell or tissue injection, injection, or transfer known to skilled artisans. In certain configurations, the apparatus can be configured for injection of chondrocytes expressing type II collagen into a nucleus pulposus of an intervertebral disc, an annulus of an intervertebral disc, an endplate of an intervertebral disc or a combination thereof.

In various embodiments of the present teachings, methods are provided for purveying to a treatment provider chondrocytes for repairing a degenerative intervertebral disc in a patient in need thereof. In various aspects, a method of these embodiments can comprise growing cadaver chondrocytes expressing type II collagen in vitro, and delivering the chondrocytes expressing type II collagen to the treatment provider. Chondrocytes expressing type II collagen of these embodiments can be, in some configurations, chondrocytes which also express high molecular weight sulfated proteoglycan. Methods of these embodiments can further comprise obtaining chondrocytes expressing type II collagen from a cadaver. In these embodiments, the cadaver chondrocytes expressing type II collagen can be obtained at various time intervals following death of the donor as described supra. Furthermore, a donor cadaver of chondrocytes expressing type II collagen can be of an age at time of death as described supra. The chondrocytes of these embodiments can be chondrocytes of tissue sources such as those described supra.

In some configurations of these methods, the chondrocytes expressing type II collagen can be purveyed to a treatment provider along with one or more biocompatible molecules, such as those described supra. In some configurations, a composition comprising the chondrocytes and one or more biocompatible molecules can be purveyed to a treatment provider. In other configurations, the chondrocytes and the one or more biocompatible molecules can be purveyed separately to a treatment provider (either simultaneously or at different times), and the treatment provider can form a composition comprising the chondrocytes and the one or more biocompatible molecules prior to, or in conjunction with, implanting the composition in a patient in need thereof.

In various embodiments, the teachings of the present application also disclose use of cadaver chondrocytes expressing type II collagen for the production of a composition for repairing a degenerative intervertebral disc in a patient in need thereof. In some configurations of these embodiments, the chondrocytes can also express high molecular weight sulfated proteoglycan. In certain configurations of these embodiments, the chondrocytes can be cadaver chondrocytes which are grown in vitro, as described supra. A composition of these embodiments can comprise a composition comprising cadaver chondrocytes expressing type II collagen and one or more biocompatible molecules such as those described supra. In addition, the time interval following death at which the chondrocytes can be removed from a donor can be a time interval as described supra, and the age of a donor cadaver at time of death can be an age as described supra. In some aspects of these embodiments, the chondrocytes can include chondrocytes removed from an annulus, chondrocytes removed from a nucleus pulposus, chondrocytes removed from an endplate of an intervertebral disc of a donor cadaver, or a combination thereof. In some other aspects of these embodiments, the chondrocytes can be chondrocytes removed from other cartilaginous, non-intervertebral disc tissue of a cadaver, such as, for example, hyaline cartilage from the nose, ears, trachea or larynx, as well as articular cartilage, costal cartilage, cartilage of an epiphyseal plate, or combinations thereof.

In various aspects, the present teachings include methods of repairing a degenerative intervertebral disc in a subject such as a human in need of treatment. These methods comprise introducing into a degenerative intervertebral disc, a composition comprising cadaver chondrocytes expressing type II collagen. In various embodiments, the cadaver chodrocytes can be cadaver chondrocytes grown in vitro, as described herein. The introducing can comprising injecting the cadaver chondrocytes through an aperture or incision in the annulus of the disc. These methods can further comprise forming a closure of the aperture or incision following the introduction of the chondrocytes into the disc. In various embodiments, the closure can withstand at least about 150 N of compression force applied to the disc, and, in some configurations, at least about 400 N of compression force. In various configurations, forming a closure can comprise applying a biocompatible glue to the surface of the annulus. In some aspects, a closure can also comprise at least one suture, i.e., forming a closure can comprise suturing the disc. In addition, in some configurations, the methods can comprise introducing an aperture or an incision into the annulus prior to introducing the composition into the intervertebral disc. In other aspects, the methods include growing cadaver chondrocytes in vitro prior to injecting them into an intervertebral disc. In various aspects, injecting the composition into a disc can comprise injecting the composition into the nucleus pulposus comprised by the disc. Furthermore, chondrocytes in certain aspects of these methods can be chondrocytes from intervertebral discs or from tissue sources other than intervertebral discs.

In various embodiments of these methods, a composition can comprise, in addition to chondrocytes, one or more biocompatible molecules, such as a macromolecule. In various aspects, each of the one or more biocompatible molecules can be fibrinogen, fibrin, thrombin, type I collagen, type II collagen, type III collagen, fibronectin, laminin, hyaluronic acid, hydrogel, pegylated hydrogel or chitosan. Furthermore, in these embodiments, the methods can further comprise forming a composition by contacting the chondrocytes with the one or more biocompatible molecules.

### BRIEF DESCRIPTION OF THE DRAWINGS

These and other features, aspects, and advantages of the present invention will become better understood with regard to the following description, appended claims and accompanying figures where:

FIG. 1 illustrates a normal intervertebral disc (left) and a herniated disc (right).

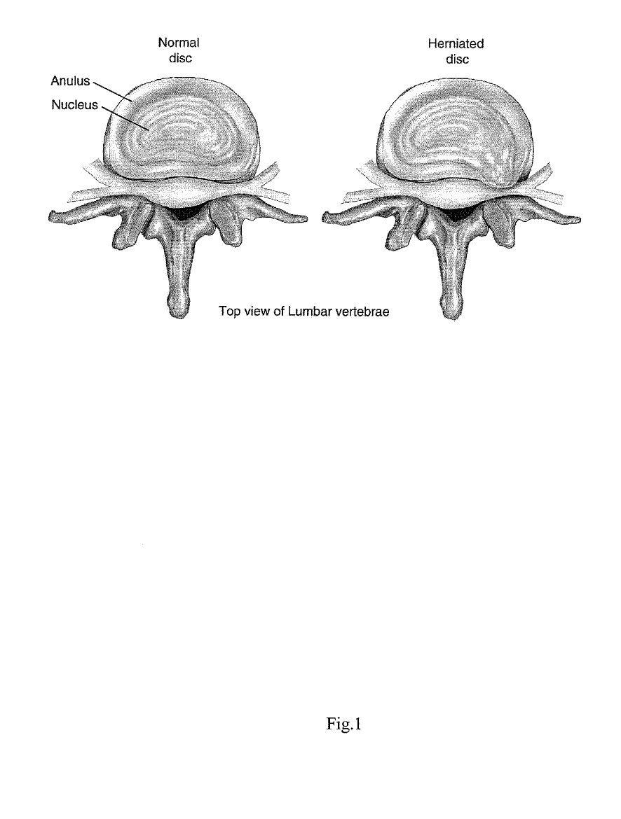

FIG. 2 illustrates freshly isolated, juvenile cartilage tissue that has been dissected to small cubes and implanted into a damaged nucleus pulposus region of an intervertebral disc in a composition which can also comprise a biocompatible molecule.

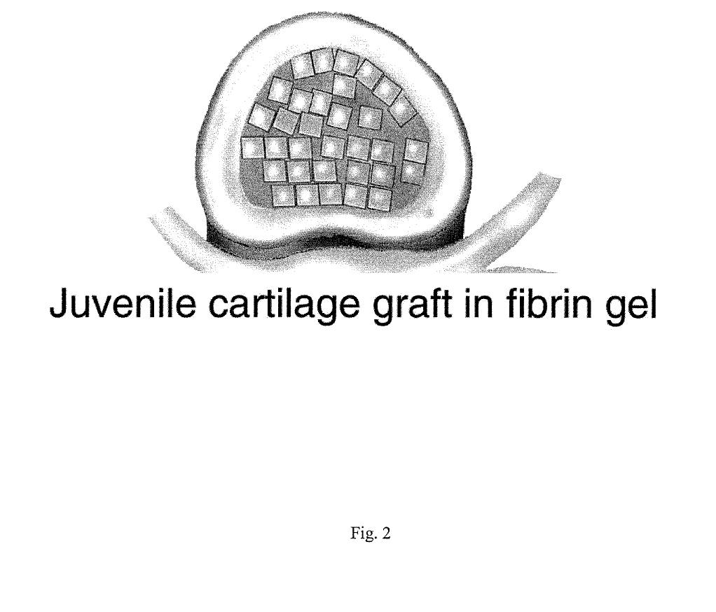

FIG. 3 illustrates isolated juvenile chondrocytes, freshly isolated or harvested from expanded in vitro cultures which can be implanted into the nucleus pulposus region of an intervertebral disc in a composition which can also comprise a biocompatible molecule.

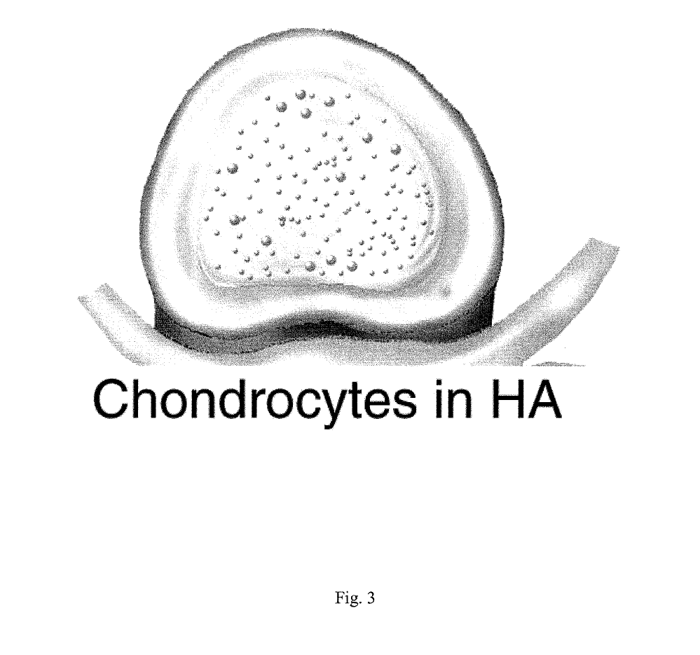

FIG. 4 illustrates the gross appearance of an intact unoperated disc harvested from the lumbar region of an adult canine.

FIG. 5 illustrates an intact nucleus pulposus and the cartilaginous endplate of the disc shown in FIG. 4.

FIG. 6 illustrates a section of an intervertebral disk that was treated with human chondrocytes 12 weeks post-injection.

FIG. 7 represents the highlighted region of FIG. 6, illustrating the newly synthesized matrix that has replaced the native nucleus 12 weeks after chondrocyte injection.

FIG. 8 illustrates the gross appearance of discs 12 weeks after chondrocyte injection.

FIG. 9 illustrates viability of chondrocytes at various holding times in thrombin-containing solution.

FIG. 10 illustrates porcine chondrocyte suspension held in thrombin solution for 5.5 hours and stained for viability analysis, 20× original magnification.

FIG. 11 illustrates a FibriJet® double barrel syringe filled with cryoprecipitated fibrinogen on one side and the chondrocyte-thrombin solution on the other.

FIG. 12 illustrates a chondrocyte-containing fibrin hydrogel formed from extruding and combining fibrinogen and chondrocyte-thrombin solutions from the FibriJet® syringe.

FIG. 13 illustrates green channel fluorescence of viable porcine chondrocytes within the fibrin matrix.

FIG. 14 illustrates MRI images of a rat tail discs.

FIG. 15 illustrates porcine disc which have received a fibrin matrix through the annulus but no closure, and subsequently placed in a material testing machine to test compression of the disc endplates.

FIG. 16 illustrates force-time and displacement-time curves of mini pig disc compression which has received a fibrin matrix through the annulus but no closure.

FIG. 17 illustrates porcine discs which have received a fibrin matrix through the annulus and a closure, either a surgical adhesive (left panel) or sutures (right panel), and subsequently placed in a material testing machine to test compression of the disc endplates.

FIG. 18 illustrates force-time and displacement-time curves of mini pig disc compression representative of annulus closure using either sutures or Bioglue® Surgical Adhesive.

FIG. 19 illustrates an X-ray image of pig spine during disc nucleus implant surgery.

FIG. 20 illustrates an X-ray image of pig spine during disc nucleus implant surgery.

### DETAILED DESCRIPTION

The present teachings describe compositions, methods and devices for repair, replacement and/or supplementation of a degenerative intervertebral disc. These methods can involve implantation or injection of chondrocytes into a degenerative disc. In addition, the present teachings also describe methods for providing chondrocytes to a treatment provider.

As used herein, the terms “degenerative intervertebral disc” and “degenerative disc” refer to an intervertebral disc exhibiting disease symptoms, abnormalities or malformations, including but not limited to herniations, disruptions, traumatic injuries, and morphological changes associated with or attributed to aging. Indications of a degenerative intervertebral disc can include, but are not limited to, brittleness of an annulus, tearing of an annulus, and shrinking of a nucleus pulposus.

In various embodiments, the present teaching include methods of repairing a degenerative disc in a human patient in need of treatment. Methods of these embodiments can comprise implanting or injecting into the intervertebral disc a composition comprising cadaver chondrocytes. As used herein, the term “cadaver chondrocytes” refers to viable chondrocytes originally comprised by a human cadaver, as well as clonal descendants of such chondrocytes, such as chondrocytes grown in vitro. Cadaver chondrocytes for use in the various aspects of the present teachings can be obtained from tissues comprising chondrocytes from a cadaver, such as cartilage tissue. Such tissues can be dissected from a cadaver using standard dissection methods well known to skilled artisans. The cartilage tissue utilized in the present teachings can comprise hyaline cartilage, such as cartilage of the nose, ears, trachea and larynx, articular cartilage, costal cartilage, cartilage of an epiphyseal plate, and combinations thereof. In various aspects, the cartilage tissue or chondrocytes can be intervertebral disc cartilage or chondrocytes, or can be cartilage or chondrocytes originating from cartilaginous tissues other than intervertebral disc tissue (herein referred to as “non-intervertebral disc chondrocytes”). Viable chondrocytes can be comprised by cartilaginous tissues in a donor cadaver for up to about two weeks after death of the donor. Accordingly, in some configurations, the time interval from the time of death of a donor (as determined, for example, by a physician or a coroner) to the time of dissection of cartilage tissue from the donor can be any time from about immediately following a pronouncement of death, to about two weeks following death, such as, without limitation, about one hour, greater than 24 hours, about two days, about three days, about four days, about five days, about six days, about seven days, about eight days, about nine days about ten days, about eleven days, about twelve days, about thirteen days, or about fourteen days after death. In addition, a donor cadaver can be of any chronological age at time of death. For example, a donor cadaver can be, at time of death, ten years old or younger, twenty years old or younger, thirty years old or younger, or forty years old or younger. A donor cadaver need not be a familial member of a recipient, or be otherwise matched immunologically. Without being limited by theory, it is believed that intervertebral cartilage comprises an “immunologically privileged” tissue, so that chondrocytes transplanted to an intervertebral disk are not subject to rejection by the recipient's immune system.

Cartilage tissue can be removed from a cadaver using any surgical or dissecting techniques and tools known to skilled artisans. Following cartilage removal from a cadaver, the cartilage tissue can be minced, dissociated into single cells or small groups of cells, and/or placed into tissue or cell culture using standard techniques and apparatuses well known to skilled artisans, such as techniques and apparatuses described in the these references. Non-limiting descriptions of methods of cartilage and chondrocyte removal and culture can be found in references such as, for example, Feder, J. et al. in: Tissue Engineering in Musculoskeletal Clinical Practice. American Academy of Orthopaedic Surgeons, 2004; Adkisson, H. D. et al., Clin. Orthop. 391S:S280-S294, 2001; and U.S. Pat. Nos. 6,235,316 and 6,645,316 to Adkisson.

Cadaver chondrocytes used in the various embodiments of the present teachings are all cadaver chondrocytes which express type II collagen. In addition, in some aspects, cadaver chondrocytes can comprise chondrocytes expressing other molecular markers such as a high molecular weight sulfated proteoglycan, such as, for example, chondroitin sulfate (Kato, Y., and Gospodarowicz, D., J. Cell Biol. 100: 477-485. 1985). Presence of such markers can be determined using materials and methods well known to skilled artisans, such as, for example, antibody detection and histological staining.

In some configurations, cadaver chondrocytes or cartilage, including cartilage tissue as well as cells, either directly extracted from a cadaver or grown in vitro, can be harvested prior to implantation or injection into a patient, using cell culture techniques and apparatuses well known to skilled artisans, such as culture methods for neocartilage described in U.S. Pat. Nos. 6,235,316 and 6,645,764 to Adkisson, and other general laboratory manuals on cell culture such as Sambrook, J. et al., Molecular Cloning: a Laboratory Manual (Third Edition), Cold Spring Harbor Laboratory Press, Cold Spring Harbor, N.Y., 2001; and Spector, D. L., et al., Culture and Biochemical Analysis of Cells, Cold Spring Harbor Laboratory Press, Cold Spring Harbor, N.Y. 1998. In vitro culture of cadaver chondrocytes can be used to increase numbers of chondrocytes which can be implanted into a patient. In addition, routine laboratory measures known to skilled artisans can be used to detect and remove non-chondrocyte cells from a cell culture, or to test a culture for the presence of biological contaminants such as microorganisms and viruses. Primary cultures established starting from cadaver chondrocytes can be grown as long as the chondrocytes remain viable and maintain their normal in vitro histological properties.

Various configurations of the present teachings include compositions comprising chondrocytes and one or more biocompatible molecules. These biocompatible molecules can include molecules that enhance survival an/or integration of implanted chondrocytes or cartilaginous tissues into an intervertebral disc. Examples of such molecules include, without limitation, fibrinogen, fibrin, thrombin, type I collagen, type II collagen, type III collagen, fibronectin, laminin, hyaluronic acid, hydrogel, pegylated hydrogel, chitosan, and combinations thereof. Various commercial formulations comprising such molecules, such as, for example, Tisseel® fibrin glue (Baxter Healthcare Corporation, Westlake Village, Calif.) can comprise a composition of the present teachings. Accordingly, a composition of the present teachings can comprise, in non-limiting example, chondrocytes grown in culture and Tisseel® fibrin glue.

In various methods of the present teachings, cadaver chondrocytes, including but not limited to cadaver chondrocytes grown in vitro and cartilage tissue maintained in tissue culture in vitro, can be implanted or injected into an intervertebral disc of a recipient patient using surgical methods and apparatuses known to skilled artisans but adapted for such use. In various configurations, chondrocytes or cartilage of the present teachings can be implanted or injected into an annulus of a degenerative intervertebral disc, a nucleus pulposus of an intervertebral disc, one or both endplates of a degenerative intervertebral disc, or a combination thereof. In certain aspects, an aperture or incision can be introduced into the annulus of an intervertebral disc. The aperture or incision can provide a path for introducing chondrocytes or cartilage tissue into a disc.

In various configurations, cells or tissue can be placed into an apparatus or device configured for transfer of chondrocytes to or from an intervertebral disc patient, such as, in non-limiting example a biopsy instrument or transplantation instrument comprising a hollow tube or needle, a syringe, a double syringe, a hollow tube, a hollow needle such as a Jamshidi needle, a Cook needle (Cook incorporated, Bloomington, Ind. USA), a cannula, a catheter, a trocar, a stylet, an obturator, or other instruments, needles or probes for cell or tissue injection, injection, or transfer known to skilled artisans. Accordingly, an apparatus of the present teachings can comprise cadaver chondrocytes as described above, as well as at least one hollow needle or tube through which the chondrocytes can be introduced into an intervertebral disc of a patient. In some configurations, the apparatus comprises a composition comprising the chondrocytes as well as at least one biocompatible molecule as described supra. These apparatuses can be configured for implanting or injecting chondrocytes into an annulus, a nucleus pulposus, and/or an endplate of a degenerative disc. Furthermore, surgical techniques such as vertebroplasty and kyphoplasty (Garfin, S. R., et al., Spine 26: 1511-1515, 2001) can be adapted for introduction of chondrocytes into a degenerative disc of a patient. In non-limiting example, an instrument for such as a bone tamp/balloon can be inserted into a degenerative intervertebral disc, and used to create or expand a space or cavity within a degenerative disc, for example in the nucleus pulposus of the disc. The balloon can be removed, and chondrocytes expressing type II collagen can then be injected into the expanded space, for example through a catheter.

In various embodiments and configurations, the present teachings also disclose methods of purveying to a treatment provider chondrocytes for repairing a degenerative intervertebral disc in a patient in need thereof. These methods can comprise obtaining chondrocytes from a cadaver, growing the chondrocytes in vitro, then delivering the chondrocytes to the treatment provider. The chondrocytes can be obtained from a cadaver using methods described supra, and can be chondrocytes which are adapted for injection into a degenerative intervertebral disc in a patient. The adaptation can comprise, in various configurations, expanding the numbers of chondrocytes through growth in vitro. Chondrocytes adapted for injection can also comprise, in certain aspects, chondrocytes which can be loosely connected or unattached to each other, and can be chondrocytes not comprised by cartilage tissue. The cadaver chondrocytes of these embodiments can be chondrocytes expressing type II collagen, as described supra, and can also be chondrocytes expressing high molecular weight sulfated proteoglycan, also as described supra. The chondrocytes can be delivered to a treatment provider, as either a chondrocytes grown in vitro and/or as cartilage tissue pieces as described supra. The treatment provider can be, in non-limiting example, a physician such as an orthopedic surgeon, or an agent or employee of the physician or a health care institution such as a hospital or outpatient clinic. Accordingly, in non-limiting example, cadaver chondrocytes can be grown in vitro, and delivered to the treatment provider via a delivery service such as, for example, a courier or an overnight shipper. Cadaver chondrocytes and/or cartilage tissue can be prepared for delivering by methods well known to skilled artisans. In some configurations, cadaver chondrocytes and/or cartilage tissue can be provided in a composition further comprising at least one biocompatible molecule as described supra. In alternative configurations, the chondrocytes and/or cartilage tissue can be packaged and sent separately from any biomolecule(s). The treatment provider can then form the composition by mixing the cells with the one or more biomolecules. In some aspects, the mixing can be done immediately prior to implanting the cells into a recipient patient.

In various aspects, the present teachings provide methods of repairing a degenerative intervertebral disc. In various configurations, these methods comprise introducing a composition comprising cadaver chondrocytes expressing type II collagen into a degenerative disc of a subject in need of treatment. In various aspects, introducing a composition can comprise injecting or implanting the composition. In various configurations, a composition can be introduced through an aperture or incision in the annulus of the disc. In various configurations, an aperture or incision can be formed prior to the introduction of the composition, for example by cutting an annulus with a scalpel, or by piercing the annulus with a hypodermic syringe needle that is operably attached to a syringe comprising the composition. A composition comprising cadaver chondrocytes can be deposited into the disc, e.g., into the nucleus pulposus of the disc, through the aperture or incision. Following administration of the composition, an aperture or incision can be closed, for example by application of a biocompatible glue such as BioGlue® Surgical Adhesive (CryoLife, Inc., Kennesaw, Ga.), which comprises albumin and a cross-linking agent (glutaraldehyde). Alternatively, an aperture or incision can be closed through the use of suturing. Such closures not only can prevent leakage of the composition, they can also withstand compressive force on the disc, which, in various configurations, can be at least about 150 N, at least about 400 N, at least 1000 N, or greater.

In various configurations, the methods can comprise obtaining cadaver chondrocytes, and growing cadaver chondrocytes in vitro prior to the injecting, utilizing methods set forth herein. In addition, the tissue origin of the chondrocytes can be intervertebral disc tissue, or non-intervertebral disc tissue such as, for example, cartilage of the nose, ears, trachea and larynx, as well as articular cartilage, costal cartilage, cartilage of an epiphyseal plate, or combinations thereof. In various aspects of these methods, the composition can further comprise one or more biocompatible molecules. A biocompatible molecule can be, for example, a polymer or biological macromolecule, such as, without limitation, fibrinogen, fibrin, thrombin, type I collagen, type II collagen, type III collagen, fibronectin, laminin, hyaluronic acid, hydrogel, pegylated hydrogel or chitosan. Accordingly, these methods can include forming the composition by contacting the chondrocytes with the one or more biocompatible molecules.

### EXAMPLES

The following examples are illustrative and are not intended to limit the scope of any claim.

### Example 1

This example illustrates transplantation and survival of human chondrocytes transplanted into canine intervertebral disc tissue.

In this example, a pilot animal study was conducted to determine whether human articular chondrocytes survive injection to produce cartilaginous matrices in experimental defects created in the intervertebral disk of adult canines. Gross morphologic and histological results obtained from this short-term pilot study (12 weeks) demonstrate that implanted chondrocytes can survive to produce cartilaginous matrices which integrate with surrounding host tissues.

Surgical Procedure: Prior to induction of anesthesia, six adult female dogs were sedated by the attending veterinarian or the veterinary technician/anesthetist using one of the following combinations: Atropine 0.05 mg/kg IM with or without Acepromizine 0.05-0.2 mg/kg IM. An 18 or 20 gauge 1¼ to 2 inch angio-catheter was placed in the cephalic saphenous or auricular vein for venous access. General anesthesia was induced with Pentothal (10-20 mg/kg IV to effect). Animals were intubated with a 7.0 mm-9.0 mm hi-low pressure cuff endotracheal tube. Anesthesia was maintained with isoflurane 2.5-4% in an air-oxygen mixture of 40-60%. The tube was connected to low-pressure continuous suction, and mechanical ventilation was initiated and maintained at 10 ml/kg tidal volume and at a rate of 8-10/minute. Crystalloids were provided at a rate of 7-10 mg/kg/hr.

Surgical exposure consisted of a 10 cm incision along the abdominal midline, followed by soft tissue dissection to permit transperitoneal exposure of the anterior lumbar spine.

Blunt dissection using a Cobb elevator and electrocautery was performed as needed to expose the anterior aspects of the L3-L4, L5-L6, and L7-S1 intervertebral disc space. Surgical defects (1×3 mm) were created through the annulus into the disc nucleus using a 16 gauge biopsy needle (Jamshidi needle) and aspiration. A significant volume of the nucleus was removed in concert with the annulus.

Human Neocartilage produced at ISTO Technologies according to U.S. Pat. Nos. 6,235,316 and 6,645,764 were enzymatically dissociated in HL-1 Serum-free Medium (Cambrex Bio Science, Walkersville, Md.) containing 60 units/ml CLS4 collagenase (Worthington, Lakewood, N.J.) and 50 units/mL hyaluronidase (Sigma, St. Louis, Mo.). The dissociated chondrocytes (derived from the articular cartilage of a six year old individual) were washed in fresh HL-1 medium and briefly exposed to 0.25% EDTA before pelleting at 500×g for 7 minutes. The cells were counted and stored in sterile cryovials until use. Chondrocyte viability was estimated to be greater than 90% by trypan blue exclusion. Six tubes were prepared each containing 2 million chondrocytes. The cells were then pelleted and the supernate removed. These samples were hand carried to the operating room on wet ice. Once defects were created, a chondrocyte suspension was prepared using 100 microliters of thrombin solution (Tisseel®, Baxter Healthcare Corporation, Westlake Village, Calif.). This step was completed immediately before mixing with an equivalent volume of the fibrin component (Tisseel®) using the Tisseel® injection device. 150-200 microliters of the cell suspension was injected into the intervertebral disk closest to the dog's tail (L7-S1 and L5-L6), whereas the highest vertebral level to be treated (L3-L4 or L4-L5) was filled with 100-150 microliters of cells or cell carrier. The cell suspension was injected at the base of the defect through a needle and withdrawn during expulsion until it began to spill out of the injection site, forming a solid gel. Two thirds of the control defects were left untreated (33%) or received fibrin carrier alone (33%). The final one-third of operated defects was treated with cells suspended in fibrin carrier as described above. Treatment at each of the levels was randomized to control for variability in disc size and location.

Following the surgical procedure, the fascia and underlying muscles were closed in an interrupted fashion using-0-Prolene and the skin approximated using Vicryl® (Ethicon, Inc. Somerville, N.J. USA) and Vetbond™ tissue adhesive (3M, St. Paul, Minn. USA). Blood loss, operative times and both intra- and peri-operative complications were recorded. Observations of ambulatory activities and wound healing were monitored daily, and all animals received analgesics after surgery.

Post-operative Care: After recovery from anesthesia, each dog was returned to its cage and housed singly for observation (daily) by veterinary technicians for any sign of adverse events related to surgery. Buprinorphine (0.01-0.02 mg/k IM or SC) was administered for relief of pain every 12 hours for the first 24 hours and prn thereafter. In general, the animals were pain free after 24 hrs.

Animal Harvest and Sample Collection: Dogs were sacrificed 12 weeks after surgery by overdose with euthanasia solution. Spines were removed, keeping the upper lumbar and sacral region intact. Musculoskeletal tissue was removed by dissection to expose the vertebral bodies for further sectioning using a band saw. Gross observation of the defects was performed using digital photography and the samples were immediately fixed in 10% neutral buffered formalin (Fisher Scientific, Fairlawn, N.J.) for 48 hrs. Samples were subsequently decalcified in 10% disodium EDTA (Sigma-Aldrich Co., St. Louis, Mo.) after four washes in PBS to remove formalin. Samples were then dehydrated in a graded alcohol series and processed using standard paraffin embedding.

Five micron sections were cut and stained with hematoxylin and eosin as well as safraninO for microscopic evaluation of the cartilaginous tissue present in control and operated intervertebral disks. Discs that were not exposed to the surgical procedures were used to establish normal histological features of the canine intervertebral disk.

Results: In general, the dogs handled the surgical procedure well, and all of the abdominal wounds healed rapidly without infection. There appeared to be no detrimental effect of multiple surgical procedures (operation at three vertebral levels in each animal) on the activity level of all dogs.

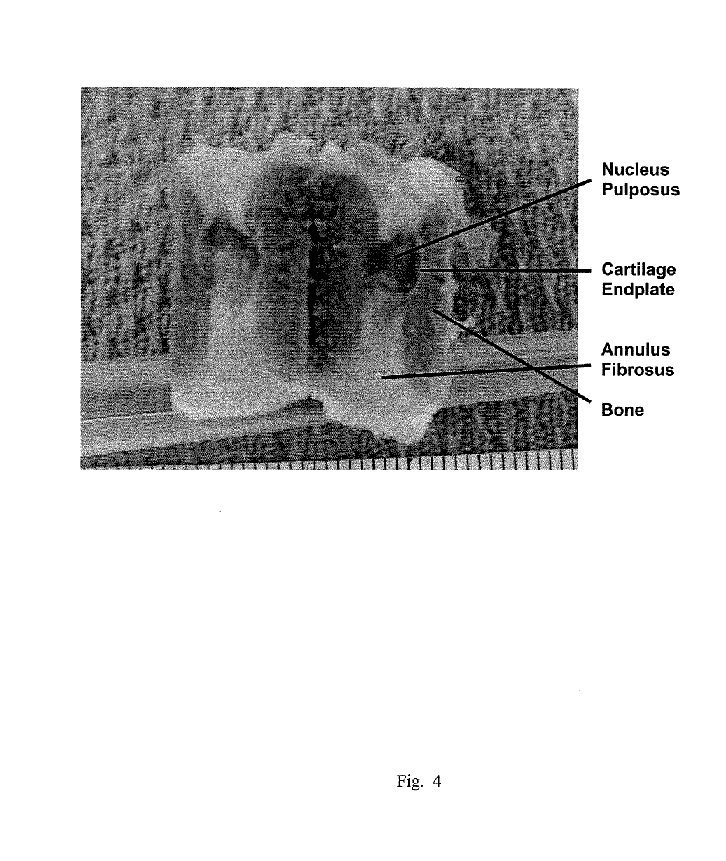

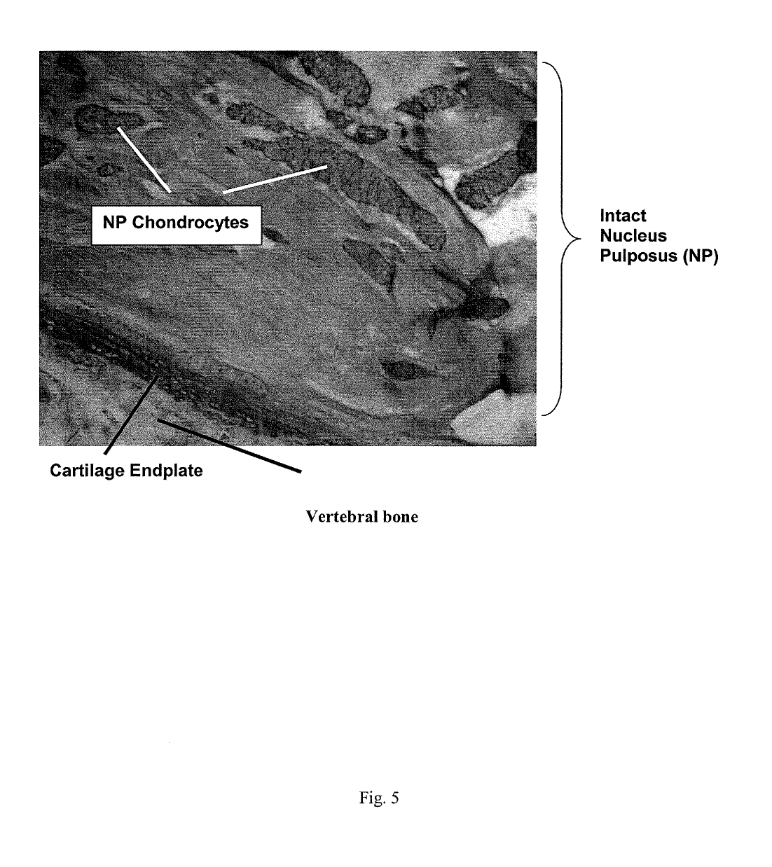

Gross macroscopic observation of the dissected vertebrae revealed normal disc structure in those discs that were not subjected to surgical intervention (FIG. 4). A glistening gelatinous center, corresponding to the nucleus pulposus, was identifiable in every case. Histological analysis revealed normal disc morphology in which the concentric rings of the annulus were observed to contain lower sulfated glycosaminoglycan content (fibrocartilaginous tissue) than the nucleus pulposus (NP) and the cartilage end plates (hyaline tissue), suggesting that surgical intervention at an adjacent level did not alter the morphological features of a disc that was not part of the procedure (FIG. 5).

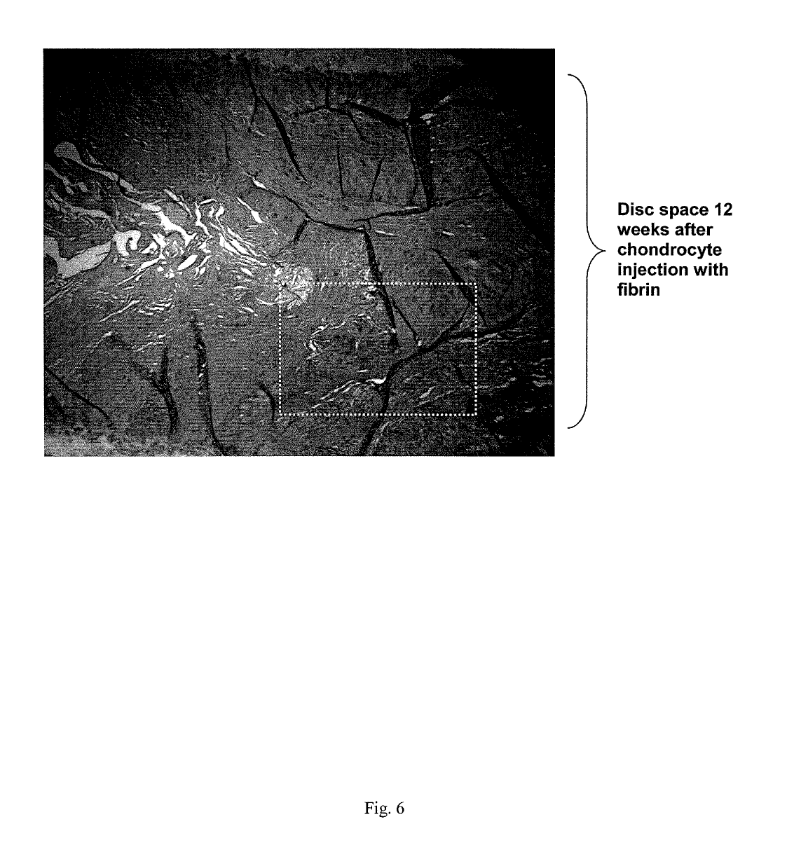

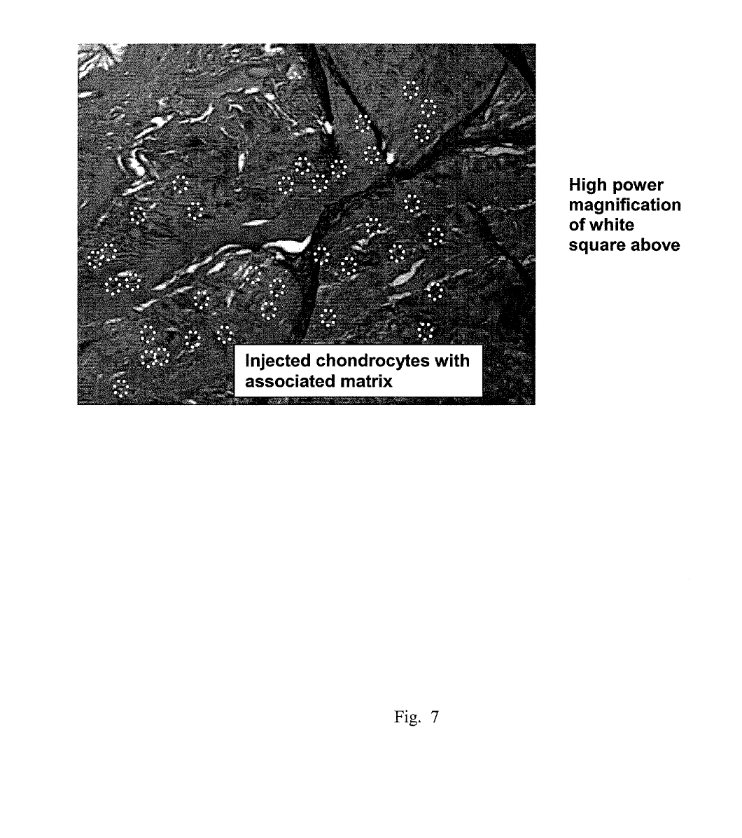

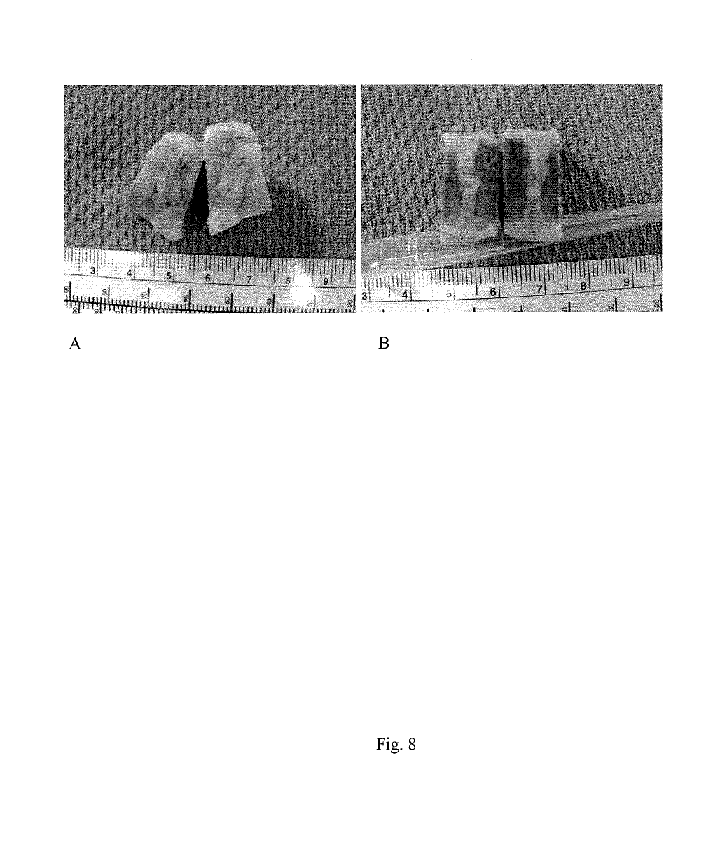

Those discs receiving neocartilage chondrocytes in fibrin glue were observed to contain viable chondrocytes in the disc space, and the injected chondrocytes had synthesized a hyaline matrix enriched in sulfated proteoglycan (FIGS. 6 and 7). Gross macroscopic observation of treated discs show viable cartilaginous tissue occupying the disc space (FIGS. 8A and B).

These results indicate that fibrin delivery to the disc space of chondrocytes derived from juvenile articular was successful and that the nature of newly synthesized tissue produced by the implanted chondrocytes appeared to be cartilaginous as determined by SafraninO staining. Most importantly, there was no histological evidence of lymphocytic infiltration into the operative site 12 weeks post-injection, suggesting that there was no immunologic rejection.

FIG. 4 illustrates the gross appearance of an intact unoperated disc harvested from the lumbar region of an adult canine. The disc is split in half to show the morphology of a normal intervertebral disk. The annulus fibrosus is the outer fibrocartilaginous structure surrounding the inner jelly-like structure or nucleus pulposus (NP). The cartilage endplate covers the surface of the upper and lower vertebral body.

FIG. 5 illustrates an intact nucleus pulposus and the cartilaginous endplate of the disc shown in FIG. 4. The section was stained with Safranin O to identify sulfated glycosaminoglycans in the NP and in the cartilage end plate. Notice that the NP chondrocytes are significantly larger than chondrocytes of the cartilage endplate and that the endplate contains greater levels of sulfated proteoglycan. Original magnification 100×

FIG. 6 illustrates a Safranin O-stained section of an intervertebral disk that was treated with human chondrocytes 12 weeks post-injection. The chondrocytes are viable and have synthesized a cartilaginous matrix that is highly enriched in sulfated glycosaminoglycans. The injected chondrocytes are much smaller than native NP chondrocytes identified in FIG. 5. The white square identifies the region shown in FIG. 7. Original magnification 40×.

FIG. 7 represents the highlighted region of FIG. 6, illustrating the newly synthesized matrix that has replaced the native nucleus 12 weeks after chondrocyte injection. The new matrix appears to be integrated well with the surrounding native tissues. Chondrocytes in this newly synthesized matrix (identified with white dotted circles) appear to be randomly distributed and of similar size to chondrocytes of the cartilaginous endplate. Original magnification 100×

FIG. 8 is in two parts. Panel A illustrates the gross appearance of a disc 12 weeks after chondrocyte injection. The native nucleus is no longer present and is replaced by newly synthesized cartilaginous tissue. Panel B illustrates the gross appearance of another disc treated in the same manner. The histological features of this disc are shown in FIGS. 6 and 7. The newly synthesized cartilaginous material produced after chondrocyte injection is expected to remodel and take on morphological features that are more characteristic of the native annulus and nucleus within 1 year after treatment.

### Example 2

This example illustrates preparation of chondrocytes. In these experiments, the joint capsule and underlying muscle were aseptically removed from a donor cadaver to expose the articular cartilage. Donors were either human (ages between 28 weeks and 3 years) or porcine (5 day old male Sinclair Minipig). The cartilage was manually recovered in small, approximately ˜1 mm thick by ˜2-3 mm rectangular pieces suitable for the digestion/isolation step. The recovered articular cartilage was placed in medium formulation HL-1 (Cambrex Corporation, East Rutherford, N.J.) supplemented with 50 μg/mL Gentamicin, 50 μg/mL L-ascorbic acid and 4 mM L-glutamine. The cells were digested free of the surrounding matrix with a purified collagenase/neutral protease, Liberase Blendzyme 2® (Roche Applied Science) at a concentration of 1.6 WU/mL. The digestion mixture was incubated at 37° C. until the digestion is complete. After digestion, any undigested material was removed by straining through a 70 μm strainer. The resulting cell suspension was then centrifuged to pellet the chondrocytes, which were then re-suspended in supplemented HL-1.

### Example 3

This example illustrates expansion of chondrocytes.

Chondrocytes digested from the matrix described in Example 2 were seeded at a density of 5×10⁶ cells/T150 flask in 30 mL of expansion medium (HL-1) supplemented with Gentamicin, L-ascorbic acid, L-glutamine, bFGF, TGF-β and 0.1% sodium hyaluronate and cultured in a 5% CO₂-37° C.-humidified incubator for 19 days. Every 3-4 days fresh medium was provided to the cells. At the first two feedings, 15 mL of expansion medium was aseptically added to each flask. At subsequent intervals, approximately 50% of the spent medium was replaced. On day 19 of culture, chondrocytes were enzymatically released from the substrate with Liberase Blendzyme 2® (0.4 WU/mL). Digestion of flasks was carried out at 5% CO₂-37° C. in a humidified incubator. After a minimum of 4 hours of digestion, the morphology of the cell clusters were observed. Digestion was determined to be complete when clusters of only 3-4 cells were seen in suspension. Once digestion was determined to be complete, chondrocytes were recovered for cryopreservation. The resulting cell suspension was then centrifuged at 350 RCF for 10-12 minutes to pellet the chondrocytes, which were then re-suspended in HL-1 medium. The centrifugation/re-suspension process was repeated to further dilute any residual enzyme activity. The re-suspended cells were counted and checked for viability.

### Example 4

This example illustrates cryopreservation of human chondrocytes.

To cryopreserve chondrocytes, a solution containing harvested chondrocytes, prepared as in Example 3, was cooled to 2-8° C. and centrifuged at 350 RCF for 10 minutes at 2-8° C., thereby pelleting the cells. The supernatant was removed and the cells re-suspended in 2-8° C. CryoStor™ freezing media to obtain a nominal concentration of 4.5×10⁷ cells/mL.

Aliquots of human cell suspension were distributed into cryovials at a volume of 1.1 mL/vial, at a density of 5×10⁷ cells/mL. The average yield of cryovials was ˜20 vials/human donor. Aliquots of porcine cell suspension are distributed into 16 cryovials at a volume of 1 mL/vial and a density of 1.3×10⁷ cells/mL. The cryovials containing the solution were allowed to equilibrate at 2-8° C. for 1 to 3 hours. After equilibration, cells were frozen in a controlled-rate freezer in liquid nitrogen vapor to a temperature of −150° C. The frozen cells were then transferred to liquid nitrogen for storage until use.

### Example 5

This example illustrates cryopreservation of porcine chondrocytes.

In this example, methods were the same as for human chondrocytes as described in Example 4, except that aliquots of porcine cell suspension were distributed into 16 cryovials at a volume of 1 mL/vial and a density of 1.3×10⁷ cells/mL.

### Example 6

This example illustrates pre-implantation stability of porcine chondrocytes.

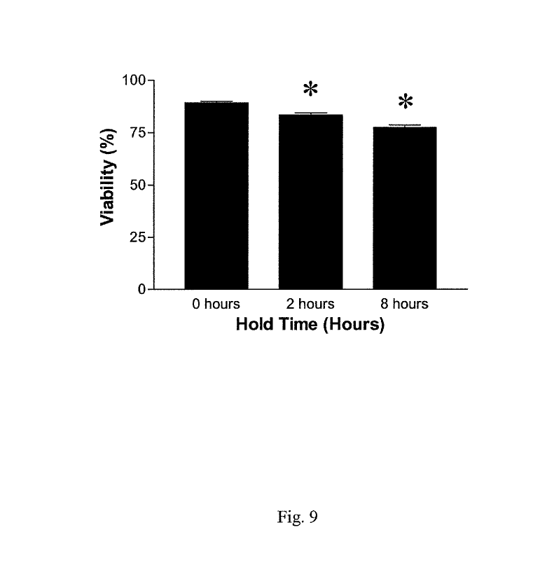

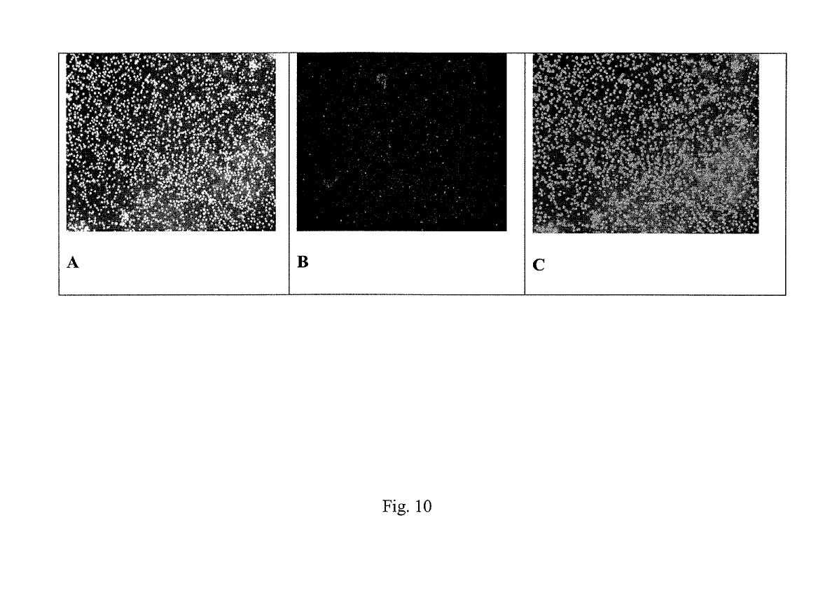

In this example, vials of frozen chondrocytes were removed from liquid nitrogen storage and rapidly thawed in a 37° C. heated water bath. The cryovials were gently swirled in the heated water bath until contents were thawed (no visible ice crystals remaining). The contents of the cryovials were transferred into a sterile tube containing 4 ml of reconstituted Thrombin (1,000 IU/ml) in saline solution yielding ˜2.45×10⁶ cells/ml. The cells were stored at room temperature (˜21° C.) and analyzed at time zero and after 2 and 8 hours for viability and total viable cell number. Viability was assessed after fluorescent staining by counting viable and non-viable cells with a Guava Technologies PCA system (FIG. 9). FIG. 10 shows porcine chondrocyte suspension held in thrombin solution for 5.5 hours and stained for viability analysis, 20× original magnification. Fluorescence in the green channel (Panel A) indicates live cells, while fluorescence in the red channel (Panel B) indicates dead cells. Combined red/green fluorescence is illustrated in Panel C. Chondrocyte viability of this cell suspension was ˜80%.

These data indicate that while viability was acceptable over the 8 hour hold period prior to implantation, there was a significant time dependent decrease in viability. We determined that preparing the cells from a frozen suspension should be done as close to the time of implantation as possible.

### Example 7

This example illustrates stability of porcine chondrocytes in a fibrin hydrogel.

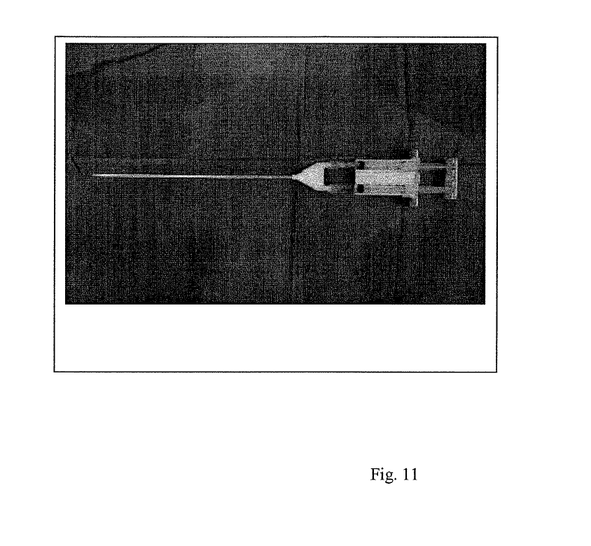

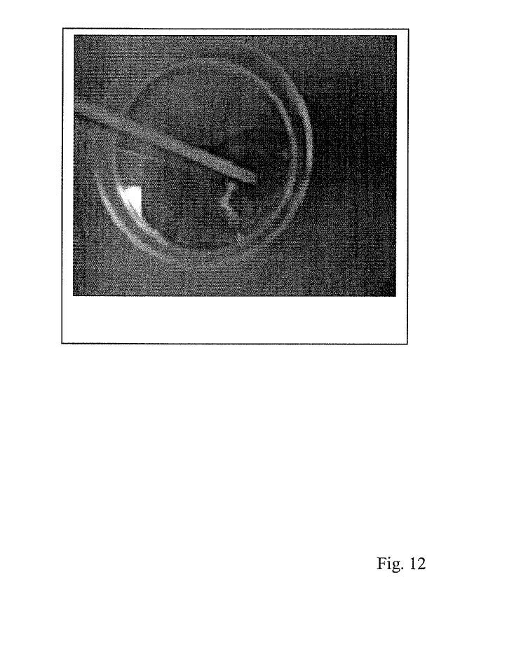

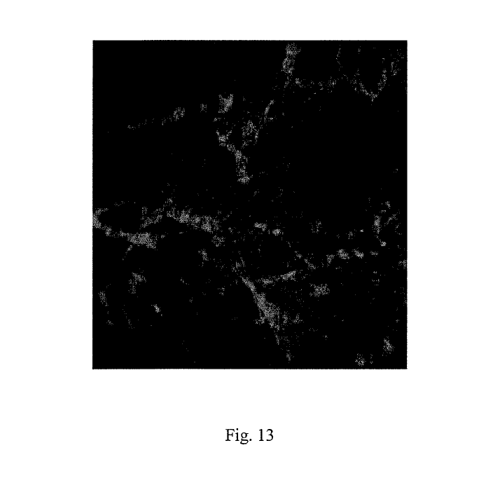

In this example, stability was assessed by staining for live and dead cells suspended in a fibrin matrix. Stained cells mixed with thrombin were mixed 1:1 with cryoprecipitated porcine fibrinogen by use of a double barrel syringe filled with cryoprecipitated fibrinogen on one side and the chondrocyte-thrombin solution on the other (FibriJet®, FIG. 11). Chondrocytes were incubated with a fluorescent live/dead stain prior to loading the syringe. The resulting hydrogel, shown in FIG. 12, was then imaged by fluorescent microscopy as illustrated in FIG. 13, which shows green channel fluorescence of viable porcine chondrocytes within the fibrin matrix. Dead cells, which fluoresce red by the assay used, were not observed in the same image field. Original image magnification was 20×. These studies indicate that chondrocytes remain viable within a hydrated fibrin clot.

### Example 8

The following example illustrates the implantation of human chondrocytes isolated from human juvenile cartilage into rat tail discs.

Chondrocytes used in this example were prepared using the same expansion, cryopreservation and reconstitution methods described in examples 2-5 above. Implantation of chondrocytes was achieved by simultaneous aspiration of nucleus cartilage and injection of the fibrin/chondrocyte mixture. The implant consisted of a ˜150 μl volume containing ˜2×10⁶ chondrocytes.

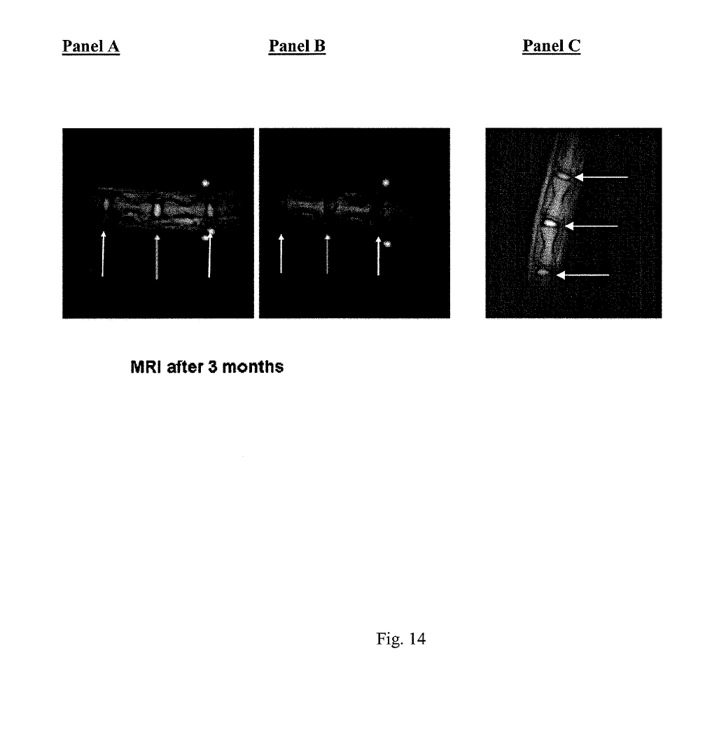

FIG. 14 illustrates replaced nucleus material 12 weeks after injection in MRI images of a rat tail discs. A: Data showing MRI intensity in an injected disc that similar to a normal disc (arrows in panel C), indicating cartilage regeneration with injected cells. In this panel, nucleus material (arrows) is replaced by human articular chondrocytes 12 weeks after injection. When compared to controls (panel C), the injected cells appear to maintain a normal disc height and morphology. B: Animals treated with fibrin alone exhibited an all black disc, suggesting no evidence of tissue regeneration. These results demonstrate that replacement of damaged nuclear material with articular chondrocytes by the disclosed methods is both feasible and practical.

### Example 9

The following example illustrates further methods to deliver and retain implanted chondrocytes into a disc nucleus within a fibrin matrix. In these studies rabbit or pig spinal columns were removed and intervertebral discs were isolated with endplate bone intact. In these experiments, an incision was made in the disc annulus, through which a SpineJet™ MicroResector (HydroCision®, Billerica, Mass.) was inserted to evacuate the nucleus material. After nucleus removal, fibrinogen and thrombin solutions were injected into the nucleus using a double barrel syringe. The incision in the annulus was either left to close on its own or was closed by one of two methods: either suturing or gluing with a bio-compatible adhesive.

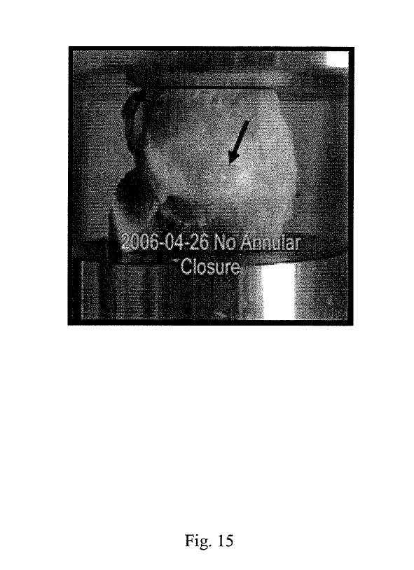

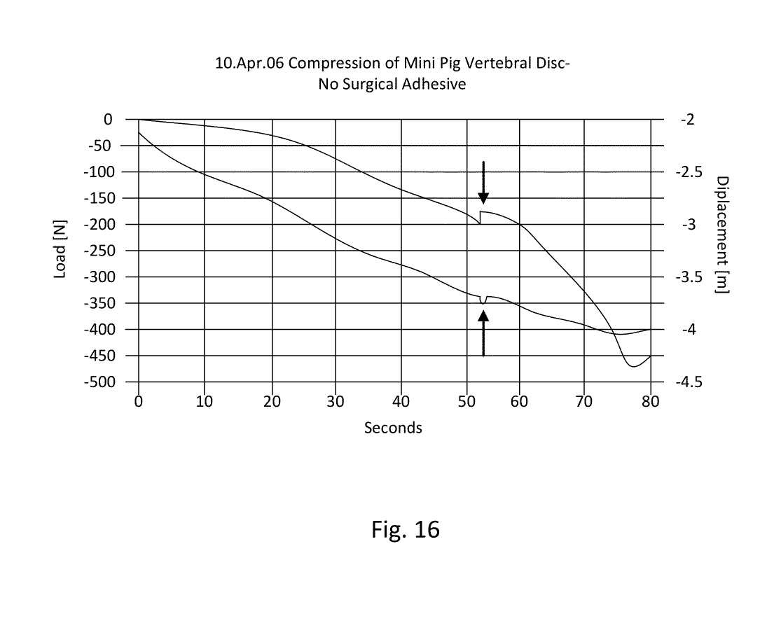

Subsequent to removal of the delivery needle from the annulus, the complete disc was placed in a material testing machine to apply compression to the disc endplates, in order to determine if the fibrin matrix would be retained under loading of the vertebral column after chondrocyte implantation. Incisions that were left untreated and allowed to close on their own failed under compressive loads as low as ˜125 N, causing the injected material to be extruded from the site of the incision (FIGS. 15 and 16). In these experiments, porcine discs were placed under compression in the testing machine. As shown in FIG. 15, subsequent to injection of a fibrin matrix through the annulus, if the incision was left untreated and allowed to close on its own. Compression under loads exceeding ˜125 N caused extrusion of the injected material from the site of the incision (arrow). In FIG. 16, force-time and displacement-time curves of mini pig disc compression are illustrated. The deflection (arrows) in both curves mark the point of failure of the annulus, with extrusion of the implanted fibrin matrix.

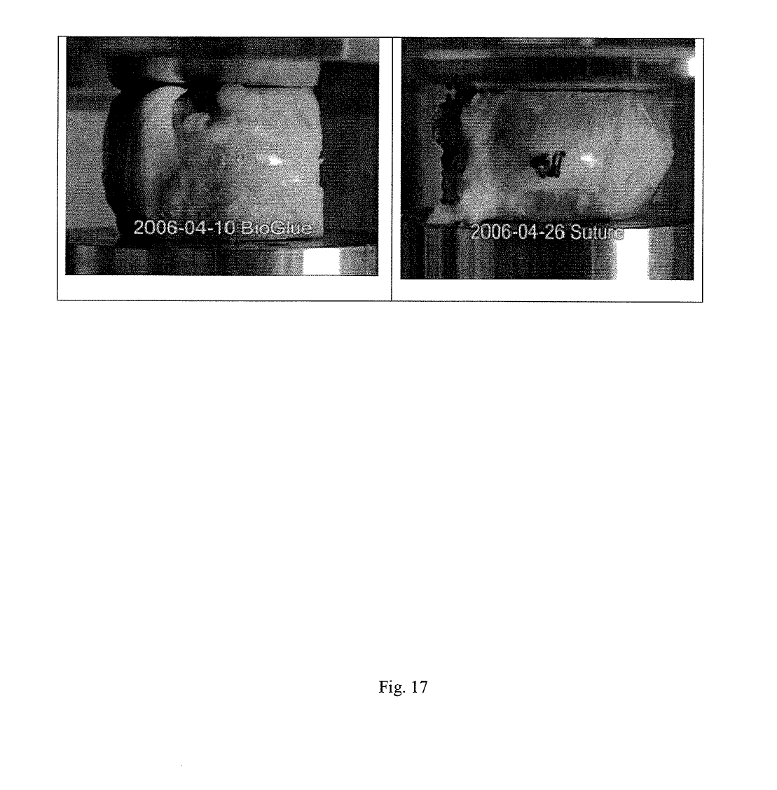

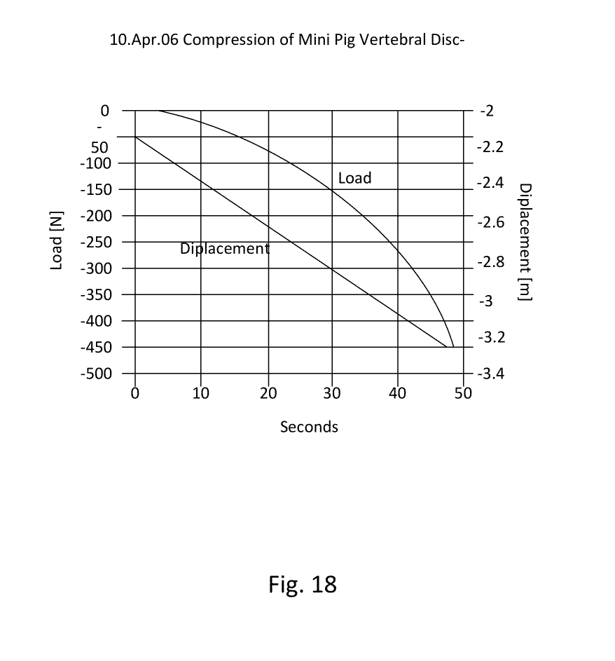

In contrast, incisions through the annulus that were closed by using either of two methods, surgical adhesive or sutures, sustained compressive loads of up to 2200 N (the limit of the testing equipment) without failure and extrusion of the implanted material (illustrated in FIGS. 17 and 18). As shown in FIG. 17, porcine discs were placed in a material testing machine under compression of the disc endplates. Subsequent to injection of a Fibrin matrix through the annulus, the incision was closed with either biological glue (Left Panel) or suturing of the annulus (Right Panel). Both the BioGlue® Surgical Adhesive (CryoLife, Inc.) and sutures prevented extrusion of the injected material under compressive loads as high as 2200 N. FIG. 18 shows force-time and displacement-time curves of mini pig disc compression representative of annulus closure using either sutures or Bioglue® Surgical Adhesive. The smooth curves and absence of material extrusion indicates that the annulus remained sealed at the maximum load of >400N applied in these experiments.

### Example 10

The following example illustrates the use of HydroCision's SpineJet™ MicroResector system in removal of nucleus pulposus in an in vivo model as well as verification of methods of delivering the fibrin matrix into the defect created by the device. In these experiments, the nucleus pulposus was removed from minipig lumbar intervertebral discs (IVD) and verified by the addition of either a contrast agent alone, or fibrin with the contrast agent. The contrast agent was used to image the IVD cavity using fluoroscopy.

The contrast agent used was Hypaque-76® NDC#0407-0778-02 (Amersham Health, Princeton, N.J.). In these experiments, the fibrinogen was extracted from porcine fresh frozen plasma using cryoprecipitation. Thrombin-JMI (King Pharmaceuticals), containing 2,000 Units/vial, was reconstituted in a mixture of 2.5 cc saline with 2.5 cc of contrast solution to yield a 5 cc solution containing 400 units thrombin/cc. Fluoroscopy enabled visualization of the IVD.

The surgery itself was initiated using a retroperitoneal approach, followed by isolation of the lumbar disc region, in which 5 IVDs were exposed. Each disc was marked with a sterile 21 g needle. Once this was accomplished, the surgeon made a 2 mm incision through the annulus into which a SpineJet™ MicroResector was inserted. Nucleus pulposus was removed during a period of not less than 2 minutes and not more than 4 minutes of use within each disc. The sequence of IVD nucleus removal was as follows:

1. Disc L2-3, which was filled with contrast agent.

2. Disc L3-4, which was filled with 0.6 cc fibrin followed by suturing the annulus (2-0 suture).

3. Disc L1-2, which was filled with 0.6 cc fibrin and non-sutured.

4. Disc L4-5, which was filled with 0.3 cc fibrin and non-sutured.

5. Disc L5-6, which was left untreated (contrast agent removed SpineJet™).

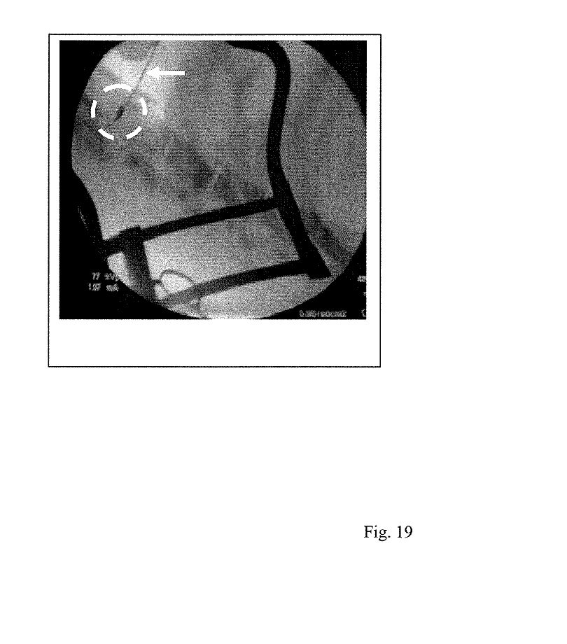

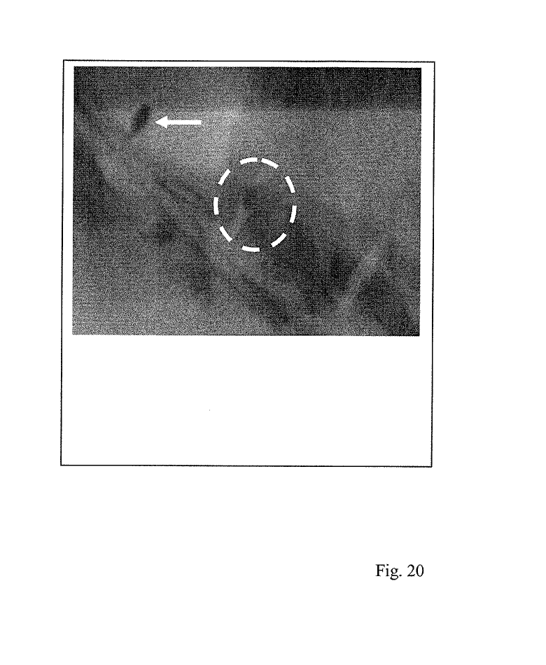

In most conditions above, contrast alone was used to determine the extent of nucleus pulposus removed while using the SpineJet™ MicroResector, as illustrated in FIG. 19. In the conditions above where fibrin was added to the cavity (2, 3 and 4), the contrast/fibrin mix was added until the solution flowed from the point in the annulus where the incision was made (FIG. 20).

FIG. 19 shows an X-ray image of pig spine during disc nucleus implant surgery. Dashed circle circumscribes the intervertebral disk. In this experiment, the nucleus was removed by use of the SpineJet™ MicroResector (HydroCision) and replaced with contrast dye (but no fibrin matrix) to verify the defect. The arrow indicates the cannula through which the dye was injected through the annulus.

FIG. 20 also shows an X-ray image of the pig spine during disc nucleus implant surgery. The dashed circle circumscribes the intervertebral disk. In this experiment, the nucleus was removed by use of the SpineJet MicroResector (HydroCision) and replaced with fibrin matrix containing contrast dye to verify the defect. The arrow indicates the nucleus replaced by contrast agent illustrated in FIG. 19.

This minipig was housed for a two week period and at the end of that time the lumbar spine was harvested and results measured using gross observations and histology of each of the 5 surgical discs. No leakage of the implanted matrix materials were observed at the time of the gross observations.

We conclude from the surgical and necropsy portions of this trial that the HydroCision SpineJet™ MicroResector is suitable for removal of the nucleus pulposus from an IVD, in the Sinclair minipig lumbar spine model system. These experiments also demonstrate that fibrin gel and sutures are adequate for retention of the fibrin gel matrix for addition of cells into the created defect.

It is to be understood that the specific embodiments of the present teachings as set forth herein are not intended as being exhaustive or limiting, and that many alternatives, modifications, and variations will be apparent to those of ordinary skill in the art in light of the foregoing examples and detailed description. Accordingly, the present teachings are intended to embrace all such alternatives, modifications, and variations that fall within the spirit and scope of the following claims.

All publications, patents, patent applications and other references cited in this application are herein incorporated by reference in their entirety as if each individual publication, patent, patent application or other reference were specifically and individually indicated to be incorporated by reference.

1. A method of implanting viable chondrocytes in a degenerative intervertebral disc in a subject, the method comprising:
a) injecting through an opening in the annulus of the degenerative intervertebral disc, into the nucleus pulposus of the disc, a composition comprising: (i) a suspension of a population of viable articular cartilage chondrocytes; (ii) thrombin; and (iii) fibrin;
b) forming a closure of the opening following injection of the cell suspension composition.

2. A method according to claim 1, wherein the composition comprises a first mixture comprising the viable population of viable articular cartilage chondrocytes suspended in a thrombin solution, and a second mixture comprising fibrinogen, the method further comprising combining the first mixture and the second mixture to form the composition immediately before or during injection.

3. A method according to claim 2, wherein the first mixture is contained in a first barrel of a double-barrel syringe, and the second mixture contained in a second barrel of the double-barrel syringe, and wherein combining the first mixture and the second mixture comprises combining the first mixture and the second mixture while extruding the mixtures simultaneously from the first and second barrels of the double-barrel syringe during injection.

4. A method according to claim 1, wherein the implanted chondrocytes are stably maintained within a fibrin matrix and regenerate cartilage in the disc for a period of at least 12 weeks.

5. A method according to claim 1, further comprising aspiration of cartilage from the nucleus pulposus simultaneously with injection of the composition.

6. A method according to claim 1, wherein the closure withstands at least about 150 N of compression force applied to the disc.

7. A method according to claim 1, wherein the closure withstands at least about 400 N of compression force applied to the disc.

8. A method according to claim 1, wherein forming a closure comprises applying a biocompatible glue to the surface of the annulus.

9. A method according to claim 1, wherein the closure comprises at least one suture.

10. A method according to claim 1, wherein injecting the composition comprises injecting the composition into the nucleus pulposus of the disc.

11. A method according to claim 1, wherein the composition further comprises one or more biocompatible molecules selected from the group consisting of fibrinogen, type I collagen, type II collagen, type III collagen, fibronectin, laminin, hyaluronic acid, hydrogel, pegylated hydrogel, chitosan and any combination thereof.

12. A method according to claim 1, wherein the viable articular chondrocytes comprise an expanded population of viable articular cartilage cadaver chondrocytes comprising an initial population of articular cartilage chondrocytes extracted from a cadaver donor, the initial population expanded in vitro in a substantially serum-free growth medium, the expanded population maintaining a chondrocytic phenotype.

13. A method of regenerating cartilaginous matrix in a degenerative intervertebral disc in a subject in need thereof, the method comprising:
a) introducing into the nucleus pulposus of the disc, through an opening in the annulus of the degenerative intervertebral disc, a fluid composition comprising viable articular chondrocytes suspended in a fibrin matrix; and
b) preventing extrusion of the fluid composition from the disc under load bearing of the disc, by closing the opening in the annulus following introduction of the fluid composition into the nucleus pulposus.

14. A method according to claim 13, further comprising forming the fluid composition immediately before or during injection.

15. A method according to claim 14, wherein forming the fluid composition comprises obtaining a first mixture comprising the viable population of viable articular cartilage chondrocytes suspended in a thrombin solution, and a second mixture comprising fibrinogen; and combining the first mixture and the second mixture to form the fluid composition immediately before or during injection.

16. A method according to claim 15, wherein the first mixture is contained in a first barrel of a double-barrel syringe, and the second mixture contained in a second barrel of the double-barrel syringe, and wherein combining the first mixture and the second mixture comprises combining the first mixture and the second mixture while extruding the mixtures simultaneously from the first and second barrels of the double-barrel syringe during injection.

17. A method according to claim 13, wherein the chondrocytes in the injected fluid composition are stably maintained within a fibrin matrix and regenerate cartilage in the disc for a period of at least 12 weeks

18. A method according to claim 13, wherein closing the opening in the annulus comprises forming a surgical closure capable of withstanding at least about 150 N of compression force applied to the disc.

19. A method according to claim 13, wherein closing the opening in the annulus comprises forming a surgical closure capable of withstanding at least about 400 N of compression force applied to the disc.

20. A method according to claim 13, wherein closing the opening in the annulus comprises sealing the opening with a biocompatible glue.

21. A method according to claim 13, wherein closing the opening in the annulus comprises forming at least one suture.

22. A method according to claim 13, further comprising aspiration of cartilage from the nucleus pulposus simultaneously with introduction of the fluid composition.

23. A method according to claim 13, wherein the composition further comprises one or more biocompatible molecules selected from the group consisting of fibrinogen, type I collagen, type II collagen, type III collagen, fibronectin, laminin, hyaluronic acid, hydrogel, pegylated hydrogel, chitosan and any combination thereof.

24. A method according to claim 13, wherein the viable articular chondrocytes comprise an expanded population of viable articular cartilage cadaver chondrocytes comprising an initial population of articular cartilage chondrocytes extracted from a cadaver donor, the initial population expanded in vitro in a substantially serum-free growth medium, and stably expressing a chondrocyte phenotype.
